(19) United States
(12) Patent Application Publication
(10) Pub. No.: US 2015/0324481 A1
(43) Pub. Date: Nov. 12, 2015

(54) Building Entity Relationship Networks from n-ary Relative Neighborhood Trees

(71) Applicant: International Business Machines Corporation, Armonk, NY (US)

(72) Inventor: W Scott Spangler, San Martin, CA (US)

(73) Assignee: International Business Machines Corporation, Armonk, NY (US)

(21) Appl. No.: 14/270,613

(22) Filed: May 6, 2014

(51) Int. Cl. G06F 17/30

(52) U.S. Cl. CPC G06F 17/30958; G06F 17/30964

(57) ABSTRACT

Entities are objects with feature values that can be thought of as vectors in N-space, where N is the number of features. Similarity between any two entities can be calculated as a distance between the two entity vectors. A similarity network can be drawn between a set of entities based on connecting two entities that are relatively near to each other in N-space. Binary relative neighborhood trees are a special type of entity relationship network, designed to be useful in visualizing the entity space. They have the intuitively simple property that the more typical entities occur at the top of the tree and the more unusual entities occur at the leaf nodes. By limiting the number of links to n+1 per node (one parent, n children), a regularized flat tree structure is created that is much easier to visualize and navigate at both a course and a fine level by domain experts.

### BACKGROUND OF THE INVENTION

1. Field of Invention

The present invention relates generally to systems and methods for building entity relationship networks. More specifically, the present invention is related to a system, method and article of manufacture for building entity relationship networks from n-ary relative neighborhood trees.

2. Discussion of Related Art

The ability to summarize and visualize a complex ontology is a well-known and long studied problem. The current best approach to solving this problem is based on creating entity similarity networks. But these networks, as they become larger, become nearly impossible for the domain expert to comprehend due to the complexity of the possible interconnections. The assumption is that the best connection to draw between entities is always the mathematically optimal one (e.g., the shortest distance between two points is a straight line). Unfortunately, this mathematically optimal diagram may present no regularized structures that make the network visually graspable for human comprehension.

Prior art techniques include using an arbitrary similarity cutoff to determine when to connect entities or some form of relative neighborhood graph. [Burke, Robin. “Knowledge-based recommender systems.” Encyclopedia of library and information systems 69. Supplement 32 (2000): 175-186.] None of these approaches make use of the position in network as an indicator of generality and, further, such representations also typically become harder to understand the larger they grow.

Embodiments of the present invention are an improvement over such prior art systems and methods.

### SUMMARY OF THE INVENTION

In this invention, a framework is presented that generates a regularized n-ary (e.g., binary) tree of entities that is approximately the same in terms of creating short paths between similar entities, but has properties that are far more intuitive to grasp visually at both the broad and detailed level. The overall intuition is to start with “typical” entities at the root of the tree, and work down toward “odd” entities at the leaves. Thus one starts with the most ordinary, general common cases and then work towards more and more unusual, atypical, and specific cases in a diagnostic hierarchy.

In one embodiment, the present invention provides a computer-implemented method comprising the steps of: receiving: (a) a target set of entities, E, (b) a set of features, F, describing entities in E, and (c) a maximum number of allowed children, n, where n>1; computing, across entities in E and features in F, a set of feature vectors comprising a feature vector for each entity in E; computing an average feature vector, A, of the set of feature vectors; identifying a root entity in E whose feature vector distance from A is smallest and assigning it as a root node in a candidate set C representing a tree of nodes; identifying another entity in E whose feature vector distance from an existing node in C is smallest and adding it as a child to that existing node when it has no more than n children, otherwise, adding it to another existing node without n children with whom its feature vector distance is smallest, where this step is repeated until all entities in E are added as children of existing nodes in C; and outputting a nodal representation of the tree.

In another embodiment, the present invention provides a non-transitory, computer accessible memory medium storing program instructions for building entity relationship networks from n-ary relative neighborhood trees comprising: computer readable program code receiving: (a) a target set of entities, E, (b) a set of features, F, describing entities in E, and (c) a maximum number of allowed children, n, where n>1; computer readable program code computing, across entities in E and features in F, a set of feature vectors comprising a feature vector for each entity in E; computer readable program code computing an average feature vector, A, of the set of feature vectors; computer readable program code identifying a root entity in E whose feature vector distance from A is smallest and assigning it as a root node in a candidate set C representing a tree of nodes; computer readable program code identifying another entity in E whose feature vector distance from an existing node in C is smallest and adding it as a child to that existing node when it has no more than n children, otherwise, adding it to another existing node without n children with whom its feature vector distance is smallest, where this step is repeated until all entities in E are added as children of existing nodes in C; and computer readable program code outputting a nodal representation of the tree.

In yet another embodiment, the present invention provides a system for creating an n-ary entity relationship tree comprising: one or more processors; and a memory storing instructions which, when executed by the one or more processors, cause the one or more processors to: receive: (a) a target set of entities, E, (b) a set of features, F, describing entities in E, and (c) a maximum number of allowed children, n, where n>1; computing, across entities in E and features in F, a set of feature vectors comprising a feature vector for each entity in E; identify a root entity in E whose feature vector distance from A is smallest and assigning it as a root node in a candidate set C representing a tree of nodes; identifying another entity in E whose feature vector distance from an existing node in C is smallest and adding it as a child to that existing node when it has no more than n children, otherwise, adding it to another existing node without n children with whom its feature vector distance is smallest, where this step is repeated until all entities in E are added as children of existing nodes in C; and a display for outputting a nodal representation of the tree.

In another embodiment, the present invention provides a method for creating an n-ary entity relationship tree comprising a set of nodes representing a set of entities, with each node in the tree having at most n children, where n>1, and the entities being described by a shared set of features and a set of feature vectors, the method comprising: (a) selecting and adding an entity as a root node of the tree based on identifying a typical entity, where the typical entity has a feature vector distance that is nearest to an average feature vector in the feature space; (b) selecting and adding the next node of the tree by selecting another entity not currently in the tree, the next node being the one with the closest feature vector distance to those nodes in the tree that do not yet have n children; (c) repeating step (b) until all entities are included as nodes in the tree; and (d) when all entities have been used to create nodes in the tree, then outputting, to a display, the resulting n-ary entity relationship tree.

### BRIEF DESCRIPTION OF THE DRAWINGS

The present disclosure, in accordance with one or more various examples, is described in detail with reference to the following figures. The drawings are provided for purposes of illustration only and merely depict examples of the disclosure. These drawings are provided to facilitate the reader's understanding of the disclosure and should not be considered limiting of the breadth, scope, or applicability of the disclosure. It should be noted that for clarity and ease of illustration these drawings are not necessarily made to scale.

FIG. 1 depicts a non-limiting example of a method associated with an embodiment of the present invention.

FIG. 2 illustrates a non-limiting example output (depicting a tree comprising a plurality of nodes) as per the teachings of the present invention.

FIG. 3 depicts a non-limiting example of a system implementing the method of the present invention.

### DESCRIPTION OF THE PREFERRED EMBODIMENTS

While this invention is illustrated and described in a preferred embodiment, the invention may be produced in many different configurations. There is depicted in the drawings, and will herein be described in detail, a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the present invention.

Note that in this description, references to “one embodiment” or “an embodiment” mean that the feature being referred to is included in at least one embodiment of the invention. Further, separate references to “one embodiment” in this description do not necessarily refer to the same embodiment; however, neither are such embodiments mutually exclusive, unless so stated and except as will be readily apparent to those of ordinary skill in the art. Thus, the present invention can include any variety of combinations and/or integrations of the embodiments described herein.

Details of the Methodology

First, the basic approach is described which can be applied whenever there is a set of homogeneous entities described by a free form text description, numeric feature vectors, or a distance matrix. Then, a detailed algorithm is disclosed to implement this approach and produce the network with the desired properties.

High Level Description

The process of building an entity tree begins with finding the root node. This is selected to be the entity that is “most typical” in the feature space of all entities. At each subsequent step in the tree generation process, a node that is “nearest” to any node in the tree is selected, where the selected node does not already have its full complement of children. For example, if the tree to be generated is a binary tree, then the next node to be added can only be a child of a node that does not already have two children. This process of adding next best entities to the tree continues until all entities are placed in the tree.

The following is a detailed description of this algorithm.

Detailed Algorithm.

Given a small input target set of entities, E, a set of features that describe the entities, F, and a maximum number of children at each node, n:
1. Create a set of feature vectors across all entities in E and features in F. One vector per entity, with one feature for each position in each vector. One example of how feature vectors might be created is through looking at the text documents describing each entity and using the words in those documents as features and the number of times each word occurs as the feature values. A non-limiting example of how documents may be represented in a vector space model is provided in U.S. Pat. No. 8,606,815, also assigned to International Business Machines Corporation. In such a representation, each document is represented as a vector of weighted frequencies of the document features (words and/or phrases).
2. Find the average feature vector, A, across all entity feature vectors.
3. Choose as the first (root) node, the entity in E whose distance is smallest from A. This is the most typical entity. This is the first node in the tree. Add this node to the candidate set C. If more than one node has the smallest value, then choose one of the smallest distance nodes at random.
4. To find the next node in the tree (e) compare all remaining entities in E (i.e., those not yet in the tree) to all nodes in the candidate set by distance. Find the entity not in the tree with the shortest distance to a node in the candidate set, C. Add a parent child link between c (parent) and the new node e (child).
5. Add e to the candidate set, C.
6. Remove e from E.
7. If c now has n children (after the addition of e as a child of c), then remove c from the candidate set C.
8. Halt when all entities in E are added somewhere in the tree.
9. Go to step 4.

To summarize the above-mentioned algorithm, first, each entity is described as a vector in the feature space. Each vector describes the entity in terms of the features that occur whenever that entity is present. The more frequent the entity co-occurrence, the larger the feature value. An average feature vector, A, is created which represents the average of all features across all entities.

To begin building the tree, a root node is first selected. The entity which is most typical, taken to be the one whose feature vector is closest to the average, A, is chosen as the root. To find the next node in the tree, a determination is made as to which node is closest to the root node among all the other nodes. This node then becomes a child of the root node.

The next node of the tree (the third node) could either be a child of the root node or a child of the other node already in the tree. Distances are compared and the node that is closest to either of the two nodes already in the tree is chosen and added as a child of the node that is closest.

At this point, let us imagine that the root node has two children. The next node chosen to be added to the tree cannot be added to the root node if the tree is binary (because each node is allowed only two children). Therefore the fourth node in the tree (in this case) can only be added to one of the two existing child nodes. Again, the node that is closest to one of these two nodes is chosen.

This process continues until all the nodes are added somewhere in the tree.

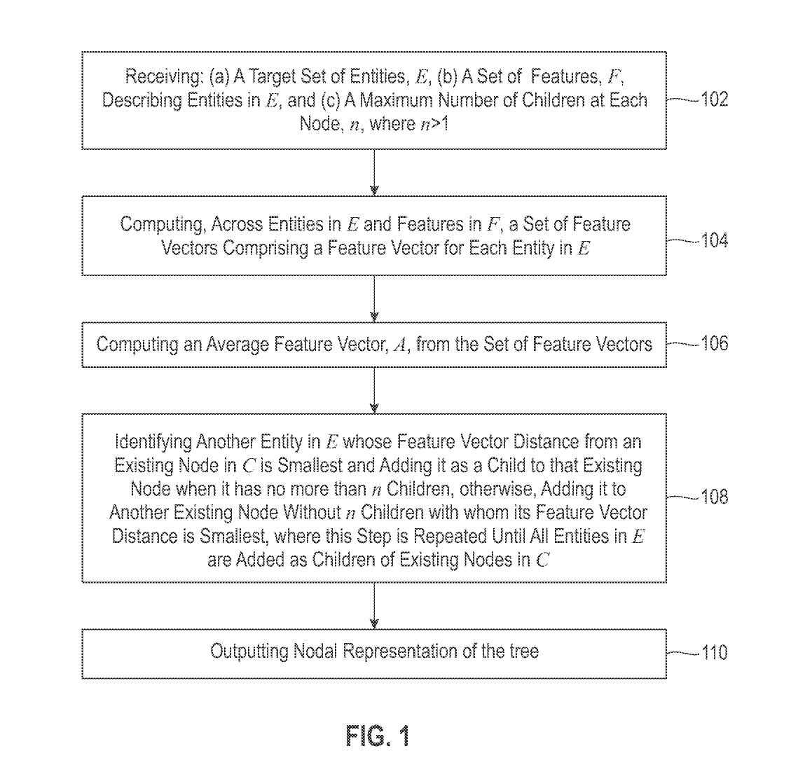

FIG. 1 depicts a non-limiting example of a method associated with an embodiment of the present invention. In this embodiment, the present invention provides a computer-implemented method comprising the steps of: receiving: (a) a target set of entities, E, (b) a set of features, F, describing entities in E, and (c) a maximum number of allowable children, n, where n>1—step 102; computing, across entities in E and features in F, a set of feature vectors comprising a feature vector for each entity in E—step 104; computing an average feature vector, A, of the set of feature vectors—step 106; identifying a root entity in E whose feature vector distance is smallest from A and assigning it as a root node in a candidate set C representing a tree; identifying another entity in E whose feature vector distance from an existing node in C is smallest and adding it as a child to that existing node when it has no more than n children, otherwise, adding it to another existing node without n children with whom its feature vector distance is smallest, where this step is repeated until all entities in E are added as children of existing nodes in C—step 108; and outputting a nodal representation of the tree—step 110.

### Example

One example of creating a binary relative neighborhood network was done around P53 kinases. The methodology used created a model of each protein kinase that is based on the Medline® abstracts that contain only that kinase and no others. The feature space of this model is the words and phrases contained in those abstracts. The distance metric is then the cosine similarity (i.e., calculation of angle between the lines that connect each point to the origin) between each kinase's centroid (average of all feature vectors for all abstracts containing the kinase). This distance matrix can then form a similarity graph which can be visualized and reasoned over to identify suspect p53 kinases. These can then be confirmed through experimentation. This method predicted that kinases not previously known to target p53 might indeed do so.

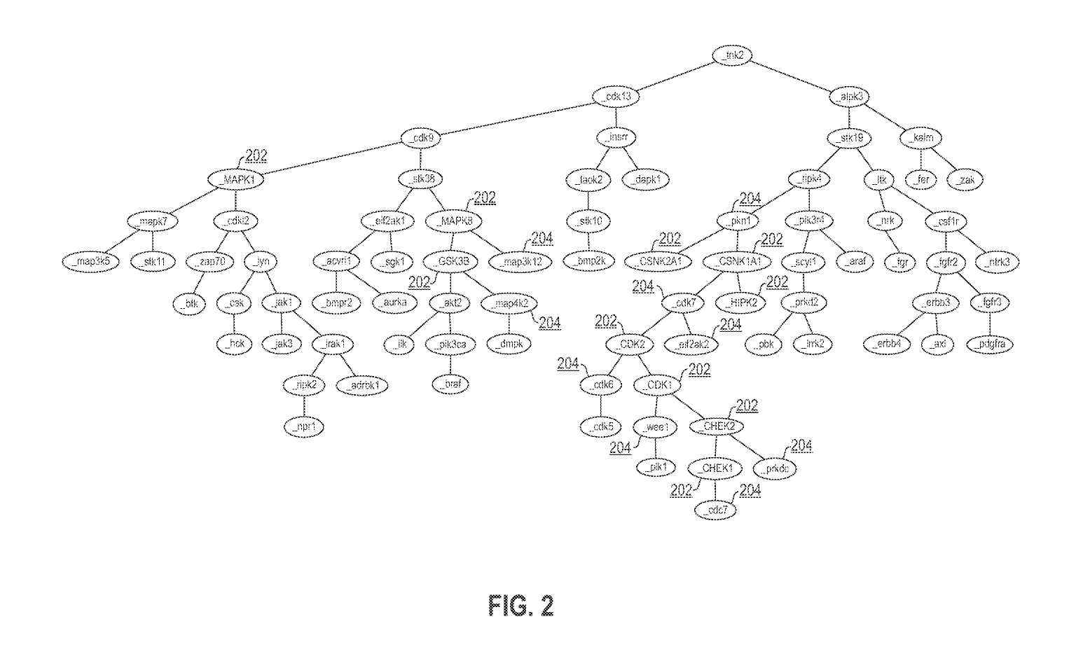

The kinase network diagram generated according to the teachings of the present invention is depicted in FIG. 2. In FIG. 2, a plurality of nodes labeled 202 represent p53 kinases, while a plurality of nodes labeled 204 represent hypothesized new P53 kinases based on their similarity to known p53 kinases.

Implementation

This invention may be implemented as a computer program, written in the Java programming language and executed with a Java virtual machine. This section includes the actual Java code used to implement the invention along with explanatory annotations.

```
import java.awt.*;
import java.awt.event.*;
import java.util.*;
import java.io.*;
import com.ibm.cv.*;
import com.ibm.cv.text.*;
import com.ibm.cv.api.*;
// The user interface for the Run Time Environment
public class ExportTree {
TextClustering tc = null;
float distances[ ][ ] = null;
Vector connections = null; // list of String[2] pairs
HashSet usedNodes = new HashSet( );
HashSet usedNodes2 = new HashSet( );
HashSet usedNodes3 = new HashSet( );
int doc[ ] = null;
String pointNames[ ] = null;
public ExportTree(TextClustering t) {
    tc = t;
    pointNames = new String[tc.ndata];
    for (int i=0; i<pointNames.length; i++) pointNames[i] = “”+(i+1);
}
public void findRootNode( ) {
    float d[ ] = ClusterView.getMeanClusterDistances(tc);
    //Util.print(d);
    int order[ ] = Index.run(d);
    int node = order[0];
    usedNodes.add(tc.clusterNames[node]);
}
public boolean findLink2( ) {
    int bestin = −1;
    int bestout = −1;
    float bestd = 100.0F;
    for (int i=0; i<tc.nclusters; i++) {
      for (int j=i+1; j<tc.nclusters; j++) {
          String a = tc.clusterNames[i];
          String b = tc.clusterNames[j];
          if (!usedNodes.contains(a) && !usedNodes.contains(b))
          continue;
          if (usedNodes.contains(b) && usedNodes.contains(a))
          continue;
          if (usedNodes3.contains(a) || usedNodes3.contains(b))
          continue;
          float d = distances[i][j];
              if (d<bestd) {
                bestd = d;
                if (usedNodes.contains(a)) {
                    bestin = i;
                    bestout = j;
                }
                else {
                    bestin = j;
                    bestout = i;
                }
              }
          }
        }
        if (bestin==−1) {
          return(false);
        }
        String s[ ] = new String[2];
        s[0] = tc.clusterNames[bestin];
        s[1] = tc.clusterNames[bestout];
        connections.add(s);
        if (usedNodes2.contains(s[0])) usedNodes3.add(s[0]);
        else usedNodes2.add(s[0]);
        System.out.println(“added connection: ” + s[0] + “-->” + s[1]);
        usedNodes.add(s[1]);
        return(true);
    }
    public void buildTree( ) {
        connections = new Vector( );
        distances = calculateAllDistances(tc);
        findRootNode( );
        int i= 1;
        while (findLink2( )) {
          System.out.println(“step ” + i);
          i++;
        }
    }
public static float[ ][ ] calculateAllDistances(KMeans k)
        { // cosine distance calculation
            // in the resulting matrix, j is always greater than i
            float result[ ][ ] = new float[k.nclusters][k.nclusters];
            float ss[ ] = new float[k.nclusters];
            for (int i=0; i<ss.length; i++)
            {
                ss[i] =
(float)Math.sqrt(Util.dotProduct(k.centroids[i],k.centroids[i]));
            }
            for (int i=0; i<result.length; i++)
            {
                for (int j=i+1; j<result.length; j++)
                {
                    float denom = ss[i]*ss[j];
                    result[i][j] =
distance(k.centroids[i],k.centroids[j],denom);
                }
            }
            return(result);
        }
      public void writeTree(String outfile) {
        try {
          PrintWriter pw = Util.openAppendFile(outfile);
          pw.println(“Tree: ” + name);
          for (int i=0; i<connections.size( )−1; i++) {
              String s[ ] = (String[ ])connections.elementAt(i);
              String node1 = “_” + cleanUp(s[0]);
              String node2 = “_” + cleanUp(s[1]);
              pw.print(node1 + “--” + node2 + “;”);
          }
          String s[ ] =
          (String[ ])connections.elementAt(connections.size( )−1);
          String node1 = s[0];
          String node2 = s[1];
          pw.println(node1 + “--” + node2 + “}”);
          pw.close( );
          } catch (Exception e) {e.printStackTrace( );}
      }
public static void main(String args[ ]) {
    ClusterHierarchy ch = ClusterHierarchy.load(args[0]);
    ExportTree x = new ExportTree(ch.getTextClustering( ));
    x.buildTree( );
    x.writeTree(args[1]);
}
```

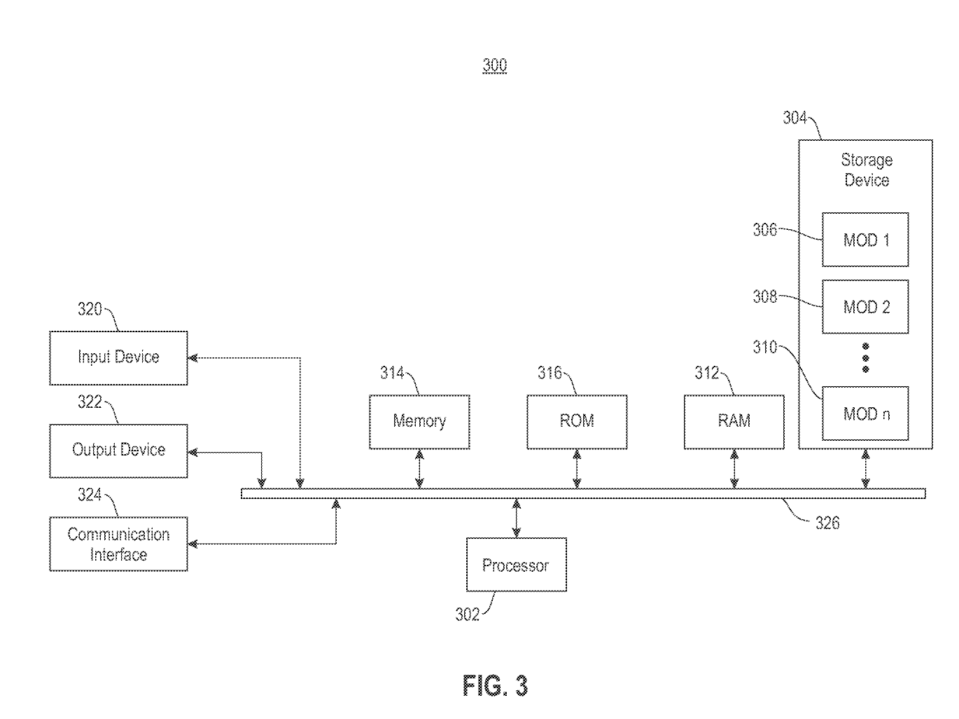

The logical operations of the various embodiments are implemented as: (1) a sequence of computer implemented steps, operations, or procedures running on a programmable circuit within a general use computer, (2) a sequence of computer implemented steps, operations, or procedures running on a specific-use programmable circuit; and/or (3) interconnected machine modules or program engines within the programmable circuits. The system 300 shown in FIG. 3 can practice all or part of the recited methods, can be a part of the recited systems, and/or can operate according to instructions in the recited non-transitory computer-readable storage media. With reference to FIG. 3, an exemplary system includes a general-purpose computing device 300, including a processing unit (e.g., CPU) 302 and a system bus 326 that couples various system components including the system memory such as read only memory (ROM) 316 and random access memory (RAM) 312 to the processing unit 302. Other system memory 314 may be available for use as well. It can be appreciated that the invention may operate on a computing device with more than one processing unit 302 or on a group or cluster of computing devices networked together to provide greater processing capability. A processing unit 302 can include a general purpose CPU controlled by software as well as a special-purpose processor.

The computing device 300 further includes storage devices such as a storage device 304 such as, but not limited to, a magnetic disk drive, an optical disk drive, tape drive or the like. The storage device 304 may be connected to the system bus 326 by a drive interface. The drives and the associated computer readable media provide nonvolatile storage of computer readable instructions, data structures, program modules and other data for the computing device 300. In one aspect, a hardware module that performs a particular function includes the software component stored in a tangible computer-readable medium in connection with the necessary hardware components, such as the CPU, bus, display, and so forth, to carry out the function. The basic components are known to those of skill in the art and appropriate variations are contemplated depending on the type of device, such as whether the device is a small, handheld computing device, a desktop computer, or a computer server.

Although the exemplary environment described herein employs the hard disk, it should be appreciated by those skilled in the art that other types of computer readable media which can store data that are accessible by a computer, such as magnetic cassettes, flash memory cards, digital versatile disks, cartridges, random access memories (RAMs), read only memory (ROM), a cable or wireless signal containing a bit stream and the like, may also be used in the exemplary operating environment.

To enable user interaction with the computing device 300, an input device 320 represents any number of input mechanisms, such as a microphone for speech, a touch-sensitive screen for gesture or graphical input, keyboard, mouse, motion input, speech and so forth. The output device 322 can also be one or more of a number of output mechanisms known to those of skill in the art. In some instances, multimodal systems enable a user to provide multiple types of input to communicate with the computing device 300. The communications interface 324 generally governs and manages the user input and system output. There is no restriction on the invention operating on any particular hardware arrangement and therefore the basic features may easily be substituted for improved hardware or firmware arrangements as they are developed.

Logical operations can be implemented as modules configured to control the processor 302 to perform particular functions according to the programming of the module. FIG. 3 also illustrates modules MOD 1 306, MOD 2 308 through MOD n 310, which are modules controlling the processor 302 to perform particular steps or a series of steps. These modules may be stored on the storage device 304 and loaded into RAM 312 or memory 314 at runtime or may be stored as would be known in the art in other computer-readable memory locations.

Modules MOD 1 306, MOD 2 308 and MOD 3 310 may, for example, be modules controlling the processor 302 to perform the following steps: (a) receiving: (1) a target set of entities, E, (2) a set of features, F, describing entities in E, and (3) a maximum number of allowable children, n, where n>1; (b) computing, across entities in E and features in F, a set of feature vectors comprising a feature vector for each entity in E; (c) computing an average feature vector, A, of the set of feature vectors; (d) identifying a root entity in E whose feature vector distance from A is smallest and assigning it as a root node in a candidate set C representing a tree of nodes; (e) identifying another entity in E whose feature vector distance from an existing node in C is smallest and adding it as a child to that existing node when it has no more than n children, otherwise, adding it to another existing node without n children with whom its feature vector distance is smallest, where this step is repeated until all entities in E are added as children of existing nodes in C; and (f) outputting nodal representation of the tree.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### CONCLUSION

A system and method has been shown in the above embodiments for the effective implementation of a system, method and article of manufacture for building entity relationship networks from n-ary relative neighborhood trees. While various preferred embodiments have been shown and described, it will be understood that there is no intent to limit the invention by such disclosure, but rather, it is intended to cover all modifications falling within the spirit and scope of the invention, as defined in the appended claims. For example, the present invention should not be limited by software/program, computing environment, or specific computing hardware.

1. A computer-implemented method comprising:
receiving: (a) a target set of entities, E, (b) a set of features, F, describing entities in E, and (c) a maximum number of allowed children, n, where n>1;
computing, across entities in E and features in F, a set of feature vectors comprising a feature vector for each entity in E;
computing an average feature vector, A, of said set of feature vectors;
identifying a root entity in E whose feature vector distance from A is smallest and assigning it as a root node in a candidate set C representing a tree of nodes;
identifying another entity in E whose feature vector distance from an existing node in C is smallest and adding it as a child to that existing node when it has no more than n children, otherwise, adding it to another existing node without n children with whom its feature vector distance is smallest, where this step is repeated until all entities in E are added as children of existing nodes in C; and
outputting a nodal representation of said tree.

2. The computer-implemented method of claim 1, wherein said entities in E are any of, or a combination of, the following: a biological entity and a chemical entity.

3. The computer-implemented method of claim 2, wherein said biological and/or chemical entities are selected from the group consisting of human genes and proteins.

4. The computer-implemented method of claim 1, wherein said set of features F are obtained based on execution of a query in a database.

5. The computer-implemented method of claim 1, wherein said tree is a binary tree.

6. The computer-implemented method of claim 1, wherein when the feature vector distance between a first entity in E and an existing parent node in C is equal to the feature vector distance between a second entity in E and the existing parent node in C, the computer-implemented method randomly picks either the first entity or the second entity to add to C.

7. The computer-implemented method of claim 1, wherein feature vectors are created by accessing a set of documents describing each entity and using words in said set of documents as features, with the number of times each word occurs in a given document being assigned as feature values of a feature vector associated with that given document.

8. A non-transitory, computer accessible memory medium storing program instructions for building entity relationship networks from n-ary relative neighborhood trees comprising:
computer readable program code receiving: (a) a target set of entities, E, (b) a set of features, F, describing entities in E, and (c) a maximum number of allowed children, n, where n>1;
computer readable program code computing, across entities in E and features in F, a set of feature vectors comprising a feature vector for each entity in E;
computer readable program code computing an average feature vector, A, of said set of feature vectors;
computer readable program code identifying a root entity in E whose feature vector distance from A is smallest and assigning it as a root node in a candidate set C representing a tree of nodes;
computer readable program code identifying another entity in E whose feature vector distance from an existing node in C is smallest and adding it as a child to that existing node when it has no more than n children, otherwise, adding it to another existing node without n children with whom its feature vector distance is smallest, where this step is repeated until all entities in E are added as children of existing nodes in C; and
computer readable program code outputting a nodal representation of said tree.

9. The non-transitory, computer accessible memory medium of claim 8, wherein said medium comprising computer readable program code identifying when the feature vector distance between a first entity in E and an existing parent node in C is equal to the feature vector distance between a second entity in E and the existing parent node in C, computer readable program code randomly picking either the first entity or the second entity to add to C.

10. The non-transitory, computer accessible memory medium of claim 8, wherein said medium further comprises computer readable program code executing a query and obtaining said set of features F.

11. The non-transitory, computer accessible memory medium of claim 8, wherein said medium further comprises:
computer readable program code formulating a query; and
computer readable program code accessing a remote database and obtaining said set of features F based on the execution of said formulated query.

12. The non-transitory, computer accessible memory medium of claim 8, wherein said medium further comprises computer readable program code creating feature vectors based on accessing a set of documents describing each entity and using words in said set of documents as features, with the number of times each word occurs in a given document being assigned as feature values of a feature vector associated with that given document.

13. A system for creating an n-ary entity relationship tree comprising:
one or more processors; and
a memory storing instructions which, when executed by the one or more processors, cause the one or more processors to:
receive: (a) a target set of entities, E, (b) a set of features, F, describing entities in E, and (c) a maximum number of allowed children, n, where n>1;
computing, across entities in E and features in F, a set of feature vectors comprising a feature vector for each entity in E;
compute an average feature vector, A, of said set of feature vectors;
identify a root entity in E whose feature vector distance from A is smallest and assigning it as a root node in a candidate set C representing a tree of nodes;
identifying another entity in E whose feature vector distance from an existing node in C is smallest and adding it as a child to that existing node when it has no more than n children, otherwise, adding it to another existing node without n children with whom its feature vector distance is smallest, where this step is repeated until all entities in E are added as children of existing nodes in C; and
a display for outputting a nodal representation of said tree.

14. The system of claim 13, wherein said entities in E are any of, or a combination of, the following: a biological entity and a chemical entity.

15. The system of claim 14, wherein said biological and/or chemical entities are selected from the group consisting of human genes and proteins.

16. The system of claim 13, wherein said system further comprises a database and said set of features F are obtained based on execution of a query in said database.

17. The system of claim 13, wherein said tree is a binary tree.

18. The system of claim 13, wherein when the feature vector distance between a first entity in E and an existing parent node in C is equal to the feature vector distance between a second entity in E and the existing parent node in C, the memory stores instructions, which when executed by the processor randomly picks either the first entity or the second entity to add to C.

19. The system of claim 13, wherein said feature vectors are created by accessing a set of documents describing each entity and using words in said set of documents as features, with the number of times each word occurs in a given document being assigned as feature values of a feature vector associated with that given document.

20. A method for creating an n-ary entity relationship tree comprising a set of nodes representing a set of entities, with each node in the tree having at most n children, where n>1, and the entities being described by a shared set of features and a set of feature vectors, the method comprising:
a) selecting and adding an entity as a root node of the tree based on identifying a typical entity, where the typical entity has a feature vector distance that is nearest to an average feature vector in the feature space;
b) selecting and adding the next node of the tree by selecting another entity not currently in the tree, the next node being the one with the closest feature vector distance to those nodes in the tree that do not yet have n children;
c) repeating step (b) until all entities are included as nodes in the tree; and
d) when all entities have been used to create nodes in the tree, then outputting, to a display, the resulting n-ary entity relationship tree.

21. The method of claim 20, wherein said entities are any of, or a combination of, the following: a biological entity and a chemical entity.

22. The method of claim 21, wherein said biological and/or chemical entities are selected from the group consisting of human genes and proteins.

23. The method of claim 20, wherein said set of features F are obtained based on execution of a query in a database.
